(19) United States
(12) Patent Application Publication
(10) Pub. No.: US 2015/0110897 A1
(43) Pub. Date: Apr. 23, 2015

(54) Sperm Protein as a Detection Biomarker of Early Stage Ovarian Cancer

(71) Applicants: Wijbe Martin Kast, La Canada, CA (US); Maurizio Chiriva-Internati, Lubbock, TX (US)

(72) Inventors: Wijbe Martin Kast, La Canada, CA (US); Maurizio Chiriva-Internati, Lubbock, TX (US)

(73) Assignees: University of Southern California, Los Angeles, CA (US); Texas Tech University System, Lubbock, TX (US)

(21) Appl. No.: 14/345,208

(22) PCT Filed: Sep. 12, 2012
(86) PCT No.: PCT/US2012/054914

§ 371 Date: Oct. 17, 2014

Related U.S. Application Data
(60) Provisional application No. 61/535,309, filed on Sep. 15, 2011.

(51) Int. Cl. G01N 33/574; C12Q 1/68

(52) U.S. Cl. CPC G01N 33/57449; C12Q 1/6886; C12Q 2600/158; G01N 2333/705; C12Q 2600/118
USPC 424/649; 435/6.11; 506/9; 435/6.12; 436/501; 506/7; 435/7.21; 514/34; 506/16; 506/18; 514/492

(57) ABSTRACT

This disclosure provides a method for determining if a patient is likely to, or not likely to, experience ovarian cancer utilizing SP17 as a biomarker. It also provides methods and therapies to treat patients identified as at risk for ovarian cancer or alternatively, as identified as having a poorer prognosis.

### CROSS-REFERENCE TO RELATED APPLICATIONS

This application claims priority under 35 U.S.C. §119(e) to U.S. provisional application Ser. No. 61/535,309, filed Sep. 15, 2011, the contents of which is hereby incorporated by reference into the present disclosure.

### BACKGROUND

Throughout this disclosure, various publications, patents and published patent specifications are referenced by an identifying citation or alternatively, by an Arabic numeral. The full bibliographic information for these citations is found in the reference section, immediately preceding the claims. The disclosures of all publications, patents and published patent specifications referenced in the text or by an Arabic numeral are hereby incorporated by reference into the present disclosure to more fully describe the state of the art to which this invention pertains.

Ovarian cancer is one of the most serious health concerns for women, ranking fifth in the leading causes of cancer-related deaths in females and second among all gynecologic malignancy-related deaths. (1) At present, serum CA125 is the most extensively used biomarker to discriminate between benign and malignant ovarian lesions, detect relapse and monitor response to treatment. (2) However, it displays limitations since up to 50% of early-stage and 10% of advanced stage ovarian cancer have normal CA125 levels. (2, 3) Because the disease's lethality has not significantly changed in the past ten years (4), there is an urgent need for innovative tools for diagnosis and prognosis of ovarian cancer.

A test able to detect the disease in the early, asymptomatic stage would save most patients, but despite enormous effort, insufficient progress has been made in this direction, and there is no proof that current screening techniques reduce ovarian cancer mortality. (5) CA125 is useful to assess response to therapy and recurrence, but is unsuitable for diagnostic purposes due to its low specificity (about 20%, depending on the study). (6-8) Great efforts are being made to identify novel biomarkers for ovarian cancer, with the goal to improve the CA125 test. (9) So far, none of the identified biomarkers has been shown to achieve the levels of sensitivity and specificity required to justify their clinical application. (9, 10)

### SUMMARY

CA125 (MUC16) is the only available biomarker to monitor disease and treatment response in ovarian cancer. Applicants have previously shown that SP17, a member of the cancer/testis antigen (CTA) family, (11) is a potential biomarker that allows for the discrimination of the normal ovary from ovarian cancer cells (12, 13) and for the tracking of ovarian cancer disease in a xenograft mouse model. (14) Yet only a few studies investigated the potential and usefulness of SP17 as a tumor biomarker (14-18) and none of them evaluated SP17 expression in the peripheral blood. Here Applicants evaluated the statistical significance of free circulating serum SP17 level measurements as a prognostic and diagnostic tool in a cohort of stage I-IV ovarian cancer patients, benign ovarian lesions and healthy women. Based on Applicants' studies, it is disclosed that the tumor associated antigen, Sperm Protein 17 (SP17), is aberrantly expressed in ovarian cancers but it is absent in normal ovarian tissues making it a novel prognostic and diagnostic biomarker for ovarian cancer.

As discussed in more detail herein, Applicants analyzed SP17 expression in primary ovarian tissues from ovarian cancer patients, benign ovarian lesions and healthy subjects through RT-PCR and immunohistochemistry. SP17 and CA125 levels were measured in the sera of the same subjects by ELISA assays. From this analysis, this disclosure provides a method for determining if a patient is likely to, or not likely to, experience ovarian cancer, (i.e. at risk of developing ovarian cancer) comprising, or alternatively consisting essentially of, or yet further consisting of, determining the expression level of the SP17 gene in a sample isolated from the patient, wherein the presence of a SP17 gene expression level or a SP17 lever higher than a predetermined first value identifies the patient as more likely to experience ovarian cancer and the absence of SP17 gene expression or SP17 gene expression lower than the predetermined first value identifies the patient as not likely to experience ovarian cancer. In a further aspect, the method further comprises, or alternatively consists essentially of, or yet further consists of, determining the expression level of the CA125 gene in a sample isolated from the patient, wherein the presence of a SP17 level and a CA125 level or SP17 and CA125 levels higher than a predetermined first value identifies the patient as more likely to experience ovarian cancer; and the presence of a CA125 level or a CA125 levels higher than a predetermined first value and the absence of an SP17 level or a SP17 level lower than the predetermined first value identifies the patient as not likely to experience ovarian cancer.

Also provided is a method for determining if an ovarian cancer patient is likely to, or not likely to, experience longer or shorter overall survival, comprising, or alternatively consisting essentially of, or yet further consisting of, determining the expression level of the SP17 gene in a sample isolated from the patient, wherein the presence of a SP17 gene expression level or a lever higher than a predetermined first value identifies the patient as more likely to experience shorter overall survival and the absence of SP17 gene expression or SP17 gene expression lower than the predetermined first value identifies the patient as likely to experience longer overall survival. In a further aspect, the method further comprises, or alternatively consists essentially of, or yet further consists of, determining the expression level of the CA125 gene in a sample isolated from the patient, wherein: the presence of a SP17 level and a CA125 level or SP17 and CA125 levels higher than a predetermined first value identifies the patient as less likely to longer overall survival; and the presence of a CA125 level or a CA125 levels higher than a predetermined first value and the absence of an SP17 level or a SP17 level lower than the predetermined first value identifies the patient as more likely to longer overall survival.

### BRIEF DESCRIPTION OF THE FIGURES

FIGS. 1A and 1B show SP17 expression in primary ovarian cancer cells. FIG. 1A. PCR was performed in different samples to analyze SP17 presence in normal ovaries, benign ovarian tumor conditions, ovarian cancer patients and in a Skov-3 ovarian cancer cell line. Testis was the internal positive control. Positive band signals were detectable in the testis, ovarian cancer patients' samples and in the Skov-3 cell line. FIG. 1B. IHC on different ovarian samples. Normal ovary and benign ovarian tumor conditions were stain-free while positive brown staining is shown through all the ovarian cancer stages. Displayed pictures are representative of different tumors at all stages.

FIGS. 2A and 2B show Kaplan-Maier survival curves. The log-rank test p<0.001 for both markers.

FIGS. 3A through 3E illustrate diagnostic performance of SP17 and CA125 tests. A through D show ROC curves of SP17 and CA125. AUC of SP17 was compared with CA125 AUC in each setting by z-test. A. SP17 AUC=0.985, CA125 AUC=0.869, z-test p=0.047; B. SP17 AUC=0.988, CA125 AUC=0.969, z-test p=0.992; C. SP17 AUC=0.991, CA125 AUC=0.941, z-test p=0.997; D SP17 AUC=0.992, CA125 AUC=0.982, z-test p=0.917; E. Algorithm combining CA125 and SP17 tests for early detection of ovarian cancer.

### DETAILED DESCRIPTION

The practice of the present invention employs, unless otherwise indicated, conventional techniques of molecular biology (including recombinant techniques), microbiology, cell biology, biochemistry and immunology, which are within the skill of the art. Such techniques are explained fully in the literature for example in the following publications. See, e.g., Sambrook and Russell eds. MOLECULAR CLONING: A LABORATORY MANUAL, 3^{rd} edition (2001); the series CURRENT PROTOCOLS IN MOLECULAR BIOLOGY (F. M. Ausubel et al. eds. (2007)); the series METHODS IN ENZYMOLOGY (Academic Press, Inc., N.Y.); PCR 1: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1999)); CULTURE OF ANIMAL CELLS: A MANUAL OF BASIC TECHNIQUE (R. I. Freshney 5^{th} edition (2005)); OLIGONUCLEOTIDE SYNTHESIS (M. J. Gait ed. (1984)); Mullis et al. U.S. Pat. No. 4,683,195; NUCLEIC ACID HYBRIDIZATION (B. D. Hames & S. J. Higgins eds. (1984)); NUCLEIC ACID HYBRIDIZATION (M. L. M. Anderson (1999)); TRANSCRIPTION AND TRANSLATION (B. D. Hames & S. J. Higgins eds. (1984)); IMMOBILIZED CELLS AND ENZYMES (IRL Press (1986)); B. Perbal, A PRACTICAL GUIDE TO MOLECULAR CLONING (1984); GENE TRANSFER VECTORS FOR MAMMALIAN CELLS (J. H. Miller and M. P. Calos eds. (1987) Cold Spring Harbor Laboratory); GENE TRANSFER AND EXPRESSION IN MAMMALIAN CELLS (S. C. Makrides ed. (2003)) IMMUNOCHEMICAL METHODS IN CELL AND MOLECULAR BIOLOGY (Mayer and Walker, eds., Academic Press, London (1987)); WEIR'S HANDBOOK OF EXPERIMENTAL IMMUNOLOGY (L. A. Herzenberg et al. eds (1996)).

### DEFINITIONS

As used herein, certain terms may have the following defined meanings. As used in the specification and claims, the singular form “a,” “an,” and “the” include singular and plural references unless the context clearly dictates otherwise. For example, the term “a cell” includes a single cell as well as a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this invention. Accordingly, it is intended that the methods and compositions can include additional steps and components (comprising) or alternatively including steps and compositions of no significance (consisting essentially of) or alternatively, intending only the stated method steps or compositions (consisting of).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

The term “identify” or “identifying” is to associate or affiliate a patient closely to a group or population of patients who likely experience the same or a similar clinical response to treatment.

The term “adjuvant” cancer patient refers to a patient to which administration of a therapy or chemotherapeutic regimen has been given after removal of a tumor by surgery, usually termed adjuvant chemotherapy. Adjuvant therapy is typically given to minimize or prevent a possible cancer reoccurrence. Alternatively, “neoadjuvant” therapy refers to administration of therapy or chemotherapeutic regimen before surgery, typically in an attempt to shrink the tumor prior to a surgical procedure to minimize the extent of tissue removed during the procedure.

As used herein, the term “patient” intends an animal, a mammal or yet further a human patient. For the purpose of illustration only, a mammal includes but is not limited to a human, a simian, a murine, a bovine, an equine, a porcine or an ovine.

Sperm protein 17 (SP17) is a highly conserved mammalian protein in the testis and spermatozoa and has been characterized as a tumor-associated antigen in a variety of human malignancies.

The term “genetic marker” or “biomarker” refers to an allelic variant of a polymorphic region of a gene of interest and/or the expression level of a gene of interest.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

The term “polymorphism” refers to the coexistence of more than one form of a gene or portion thereof. A portion of a gene of which there are at least two different forms, i.e., two different nucleotide sequences, is referred to as a “polymorphic region of a gene.” A polymorphic region can be a single nucleotide, the identity of which differs in different alleles.

A “polymorphic gene” refers to a gene having at least one polymorphic region.

A “haplotype” is a set of alleles of a group of closely linked genes which are usually inherited as a unit. The term “allelic variant of a polymorphic region of the gene of interest” refers to a region of the gene of interest having one of a plurality of nucleotide sequences found in that region of the gene in other individuals.

The term “genotype” refers to the specific allelic composition of an entire cell or a certain gene and in some aspects a specific polymorphism associated with that gene, whereas the term “phenotype” refers to the detectable outward manifestations of a specific genotype.

“Expression” as applied to a gene, refers to the production of the mRNA transcribed from the gene, or the protein product encoded by the gene. The expression level of a gene may be determined by measuring the amount of mRNA or protein in a cell or tissue sample. In one aspect, the expression level of a gene is represented by a relative level as compared to a housekeeping gene as an internal control. In another aspect, the expression level of a gene from one sample may be directly compared to the expression level of that gene from a different sample using an internal control to remove the sampling error.

An “internal control” or “house keeping” gene refers to any constitutively or globally expressed gene. Examples of such genes include, but are not limited to, β-actin, the transferring receptor gene, GAPDH gene or equivalents thereof. In one aspect of the invention, the internal control gene is β-actin.

“Overexpression” or “underexpression” refers to increased or decreased expression, or alternatively a differential expression, of a gene in a test sample as compared to the expression level of that gene in the control sample. In one aspect, the test sample is a diseased cell, and the control sample is a normal cell. In another aspect, the test sample is an experimentally manipulated or biologically altered cell, and the control sample is the cell prior to the experimental manipulation or biological alteration. In yet another aspect, the test sample is a sample from a patient, and the control sample is a similar sample from a healthy individual. In a yet further aspect, the test sample is a sample from a patient and the control sample is a similar sample from patient not having the desired clinical outcome. In one aspect, the differential expression is about 1.5 times, or alternatively, about 2.0 times, or alternatively, about 2.5 times, or alternatively, about 3.0 times, or alternatively, about 5 times, or alternatively, about 10 times, or alternatively about 50 times, or yet further alternatively more than about 100 times higher or lower than the expression level detected in the control sample. Alternatively, the gene is referred to as “over expressed” or “under expressed”. Alternatively, the gene may also be referred to as “up regulated” or “down regulated”.

A “predetermined value” for a gene as used herein, is so chosen that a patient with an expression level of that gene higher than the predetermined value is likely to experience a more or less desirable clinical outcome than patients with expression levels of the same gene lower than the predetermined value, or vice-versa. Expression levels of genes, such as those disclosed in the present invention, are associated with clinical outcomes. One of skill in the art can determine a predetermined value for a gene by comparing expression levels of a gene in patients with more desirable clinical outcomes to those with less desirable clinical outcomes. In one aspect, a predetermined value is a gene expression value that best separates patients into a group with more desirable clinical outcomes and a group with less desirable clinical outcomes. Such a gene expression value can be mathematically or statistically determined with methods well known in the art.

Alternatively, a gene expression that is higher than the predetermined value is simply referred to as a “high expression”, or a gene expression that is lower than the predetermined value is simply referred to as a “low expression”.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The phrase “amplification of polynucleotides” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu, D. Y. et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

The term “isolated” as used herein refers to molecules or biological or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

When the expression level of a gene or a genetic marker or polymorphism is used as a basis for selecting a patient for a treatment described herein, the expression level or genetic marker or polymorphism is measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of the genetic marker or polymorphism in a clinical setting is a clear indication that this parameter was used as a basis for initiating, continuing, adjusting and/or ceasing administration of the treatments described herein.

The term “treating” as used herein is intended to encompass curing as well as ameliorating at least one symptom of the condition or disease. For example, in the case of cancer, a response to treatment includes a reduction in cachexia, increase in survival time, elongation in time to tumor progression, reduction in tumor mass, reduction in tumor burden and/or a prolongation in time to tumor metastasis, time to tumor recurrence, tumor response, complete response, partial response, stable disease, progressive disease, progression free survival, overall survival, each as measured by standards set by the National Cancer Institute and the U.S. Food and Drug Administration for the approval of new drugs. See Johnson et al. (2003) J. Clin. Oncol. 21(7):1404-1411.

“An effective amount” intends to indicate the amount of a compound or agent administered or delivered to the patient which is most likely to result in the desired response to treatment. The amount is empirically determined by the patient's clinical parameters including, but not limited to the stage of disease, age, gender, histology, and likelihood for tumor recurrence.

The term “clinical outcome”, “clinical parameter”, “clinical response”, or “clinical endpoint” refers to any clinical observation or measurement relating to a patient's reaction to a therapy. Non-limiting examples of clinical outcomes include tumor response (TR), overall survival (OS), progression free survival (PFS), disease free survival, time to tumor recurrence (TTR), time to tumor progression (TTP), relative risk (RR), toxicity or side effect.

The term “likely to respond” intends to mean that the patient of a genotype is relatively more likely to experience a complete response or partial response than patients similarly situated without the genotype. Alternatively, the term “not likely to respond” intends to mean that the patient of a genotype is relatively less likely to experience a complete response or partial response than patients similarly situated without the genotype.

The term “suitable for a therapy” or “suitably treated with a therapy” shall mean that the patient is likely to exhibit one or more desirable clinical outcome as compared to a patient or patients having the same disease and receiving the same therapy but possessing a different characteristic that is under consideration for the purpose of the comparison. In one aspect, the characteristic under consideration is a genetic polymorphism or a somatic mutation. In another aspect, the characteristic under consideration is an expression level of a gene or a polypeptide. In one aspect, a more desirable clinical outcome is relatively higher likelihood of or relatively better tumor response such as tumor load reduction. In another aspect, a more desirable clinical outcome is a relatively longer overall survival. In yet another aspect, a more desirable clinical outcome is a relatively longer progression free survival or time to tumor progression. In yet another aspect, a more desirable clinical outcome is a relatively longer disease free survival. In further another aspect, a more desirable clinical outcome is a relative reduction or delay in tumor recurrence. In another aspect, a more desirable clinical outcome is a relatively decreased metastasis. In another aspect, a more desirable clinical outcome is a relatively lower relative risk. In yet another aspect, a more desirable clinical outcome is relatively reduced toxicity or side effects. In some embodiments, more than one clinical outcomes are considered simultaneously. In one such aspect, a patient possessing a characteristic, such as a genotype of a genetic polymorphism, may exhibit more than one more desirable clinical outcomes as compared to a patient or patients having the same disease and receiving the same therapy but not possessing the characteristic. As defined herein, the patients are considered suitable for the therapy. In another such aspect, a patient possessing a characteristic may exhibit one or more desirable clinical outcome but simultaneously exhibit one or more less desirable clinical outcome. The clinical outcomes will then be considered collectively, and a decision as to whether the patient is suitable for the therapy will be made accordingly, taking into account the patient's specific situation and the relevance of the clinical outcomes. In some embodiments, progression free survival or overall survival is weighted more heavily than tumor response in a collective decision making.

“Having the same cancer” is used when comparing one patient to another or alternatively, one patient population to another patient population. For example, the two patients or patient populations will each have or be suffering from colon cancer.

A “complete response” (CR) to a therapy defines patients with evaluable but non-measurable disease, whose tumor and all evidence of disease had disappeared.

A “partial response” (PR) to a therapy defines patients with anything less than complete response that were simply categorized as demonstrating partial response.

“Stable disease” (SD) indicates that the patient is stable.

“Progressive disease” (PD) indicates that the tumor has grown (i.e. become larger), spread (i.e. metastasized to another tissue or organ) or the overall cancer has gotten worse following treatment. For example, tumor growth of more than 20 percent since the start of treatment typically indicates progressive disease. “Disease free survival” indicates the length of time after treatment of a cancer or tumor during which a patient survives with no signs of the cancer or tumor.

“Non-response” (NR) to a therapy defines patients whose tumor or evidence of disease has remained constant or has progressed.

“Overall Survival” (OS) intends a prolongation in life expectancy as compared to naïve or untreated individuals or patients.

“Progression free survival” (PFS) or “Time to Tumor Progression” (TTP) indicates the length of time during and after treatment that the cancer does not grow. Progression-free survival includes the amount of time patients have experienced a complete response or a partial response, as well as the amount of time patients have experienced stable disease.

“No Correlation” refers to a statistical analysis showing no relationship between the allelic variant of a polymorphic region or gene expression levels and clinical parameters.

“Tumor Recurrence” as used herein and as defined by the National Cancer Institute is cancer that has recurred (come back), usually after a period of time during which the cancer could not be detected. The cancer may come back to the same place as the original (primary) tumor or to another place in the body. It is also called recurrent cancer.

“Time to Tumor Recurrence” (TTR) is defined as the time from the date of diagnosis of the cancer to the date of first recurrence, death, or until last contact if the patient was free of any tumor recurrence at the time of last contact. If a patient had not recurred, then TTR was censored at the time of death or at the last follow-up.

“Relative Risk” (RR), in statistics and mathematical epidemiology, refers to the risk of an event (or of developing a disease) relative to exposure. Relative risk is a ratio of the probability of the event occurring in the exposed group versus a non-exposed group.

As used herein, the terms “Stage I cancer,” “Stage II cancer,” “Stage III cancer,” and “Stage IV” refer to the TNM staging classification for cancer. Stage I cancer typically identifies that the primary tumor is limited to the organ of origin. Stage II intends that the primary tumor has spread into surrounding tissue and lymph nodes immediately draining the area of the tumor. Stage III intends that the primary tumor is large, with fixation to deeper structures. Stage IV intends that the primary tumor is large, with fixation to deeper structures. See pages 20 and 21, CANCER BIOLOGY, 2^{nd} Ed., Oxford University Press (1987).

A “tumor” is an abnormal growth of tissue resulting from uncontrolled, progressive multiplication of cells and serving no physiological function. A “tumor” is also known as a neoplasm.

The term “blood” refers to blood which includes all components of blood circulating in a subject including, but not limited to, red blood cells, white blood cells, plasma, clotting factors, small proteins, platelets and/or cryoprecipitate. This is typically the type of blood which is donated when a human patient gives blood.

### DESCRIPTIVE EMBODIMENTS

The invention further provides diagnostic, prognostic and therapeutic methods, which are based, at least in part, on determination of the expression level of a gene of interest identified herein.

For example, information obtained using the diagnostic assays described herein is useful for determining if a subject is suitable for cancer treatment of a given type or is likely to experience tumor recurrence or longer overall survival. Based on the prognostic information, a doctor can recommend a therapeutic protocol, useful for reducing the malignant mass or tumor in the patient or treat cancer in the individual.

Determining whether a subject as more or less likely to experience longer or shorter overall survival, alternatively, can be expressed as identifying a subject as more likely to experience tumor recurrence or identifying a subject as less likely to experience tumor recurrence.

It is to be understood that information obtained using the diagnostic assays described herein may be used alone or in combination with other information, such as, but not limited to, genotypes or expression levels of other genes, clinical chemical parameters, histopathological parameters, or age, gender and weight of the subject. When used alone, the information obtained using the diagnostic assays described herein is useful in determining or identifying the clinical outcome of a treatment, selecting a patient for a treatment, or treating a patient, etc. When used in combination with other information, on the other hand, the information obtained using the diagnostic assays described herein is useful in aiding in the determination or identification of clinical outcome of a treatment, aiding in the selection of a patient for a treatment, or aiding in the treatment of a patient and etc. In a particular aspect, the genotypes or expression levels of one or more genes as disclosed herein are used in a panel of genes, each of which contributes to the final diagnosis, prognosis or treatment.

Provided herein is a method for determining if a patient is likely to, or not likely to, experience ovarian cancer, comprising, or alternatively consisting essentially of, or yet further consisting of, determining the expression level of the SP17 gene in a sample isolated from the patient, wherein the presence of a SP17 gene expression level or a lever higher than a predetermined first value identifies the patient as more likely to experience ovarian cancer and the absence of SP17 gene expression or SP17 gene expression lower than the predetermined first value identifies the patient as not likely to experience ovarian cancer. In a further aspect, the method further comprises, or alternatively consists essentially of, or yet further consists of, determining the expression level of the CA125 gene in a sample isolated from the patient, wherein the presence of a SP17 level and a CA125 level or SP17 and CA125 levels higher than a predetermined first value identifies the patient as more likely to experience ovarian cancer; and the presence of a CA125 level or a CA125 levels higher than a predetermined first value and the absence of an SP17 level or a SP17 level lower than the predetermined first value identifies the patient as not likely to experience ovarian cancer.

Also provided is a method for determining if an ovarian cancer patient is likely to, or not likely to, experience longer or shorter overall survival, comprising, or alternatively consisting essentially of, or yet further consisting of, determining the expression level of the SP17 gene in a sample isolated from the patient, wherein the presence of a SP17 gene expression level or a lever higher than a predetermined first value identifies the patient as more likely to experience shorter overall survival and the absence of SP17 gene expression or SP17 gene expression lower than the predetermined first value identifies the patient as likely to experience longer overall survival. In a further aspect, the method further comprises, or alternatively consists essentially of, or yet further consists of, determining the expression level of the CA125 gene in a sample isolated from the patient, wherein: the presence of a SP17 level and a CA125 level or SP17 and CA125 levels higher than a predetermined first value identifies the patient as less likely to longer overall survival; and the presence of a CA125 level or a CA125 levels higher than a predetermined first value and the absence of an SP17 level or a SP17 level lower than the predetermined first value identifies the patient as more likely to longer overall survival.

Briefly and for the purpose of illustration only, one of skill in the art can determine the predetermined values by comparing expression values of a gene in patients with more desirable clinical parameters to those with less desirable clinical parameters. In one aspect, a predetermined value is a gene expression value that best separates patients into a group with more desirable clinical parameter and a group with less desirable clinical parameter. Such a gene expression value can be mathematically or statistically determined with methods well known in the art.

Suitable samples for use in the methods of this invention include, but are not limited to a blood sample, a urine sample or a serum sample.

Methods to determine gene expression level are known in the art and briefly described herein. Non-limiting examples of these methods include a method that comprises, or alternatively consists essentially of, or yet further consists of, determining the amount of mRNA transcribed from the gene, mRNA in situ hybridization, PCR, real-time PCR, or microarray. The methods are useful in the assistance of a patient such as an animal, a mammal or yet further a human patient. For the purpose of illustration only, a mammal includes but is not limited to a simian, a murine, an ovine, an equine, a canine, a bovine, a porcine or a human patient.

As alternate embodiments of each of the above noted inventions, the suitable patient sample comprises, or alternatively consists essentially of, or yet further consists of, tissue or cells selected from non-metastatic tumor tissue, a non-metastatic tumor cell, metastatic tumor tissue or a metastatic tumor cell. In another aspect the patient sample can be normal tissue isolated adjacent to the tumor.

Antibodies directed SP17 and CA125 proteins may also be used in disease diagnostics and prognostics. Such diagnostic methods, may be used to detect abnormalities in the level of expression of the peptide, or abnormalities in the structure and/or tissue, cellular, or subcellular location of the peptide. This can be accomplished, for example, by immunofluorescence techniques employing a fluorescently labeled antibody coupled with light microscopic, flow cytometric, or fluorimetric detection. The antibodies (or fragments thereof) useful in the present invention may, additionally, be employed histologically, as in immunofluorescence or immunoelectron microscopy, for in situ detection of the peptides or their allelic variants. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine not only the presence of the subject polypeptide, but also its distribution in the examined tissue. Using the present invention, one of ordinary skill will readily perceive that any of a wide variety of histological methods (such as staining procedures) can be modified in order to achieve such in situ detection.

In one embodiment, it is necessary to first amplify at least a portion of the gene of interest prior to identifying the polymorphic region of the gene of interest in a sample. Amplification can be performed, e.g., by PCR and/or LCR, according to methods known in the art. Various non-limiting examples of PCR include the herein described methods.

Assembly PCR or Polymerase Cycling Assembly (PCA) is the artificial synthesis of long DNA sequences by performing PCR on a pool of long oligonucleotides with short overlapping segments. The oligonucleotides alternate between sense and antisense directions, and the overlapping segments determine the order of the PCR fragments thereby selectively producing the final long DNA product (See, Stemmer et al. (1995) Gene 164(1):49-53 and U.S. Pat. Nos. 6,335,160; 7,058,504 or 7,323,336)

Asymmetric PCR is used to preferentially amplify one strand of the original DNA more than the other. It finds use in some types of sequencing and hybridization probing where having only one of the two complementary stands is required. PCR is carried out as usual, but with a great excess of the primers for the chosen strand. Due to the slow amplification later in the reaction after the limiting primer has been used up, extra cycles of PCR are required (See, Innis et al. (1988) Proc Natl Acad Sci U.S.A. 85(24):9436-9440 and U.S. Pat. Nos. 5,576,180; 6,106,777 or 7,179,600). A recent modification on this process, known as Linear-After-The-Exponential-PCR (LATE-PCR), uses a limiting primer with a higher melting temperature (Tₘ) than the excess primer to maintain reaction efficiency as the limiting primer concentration decreases mid-reaction (Pierce et al. (2007) Methods Mol. Med. 132:65-85).

Colony PCR uses bacterial colonies, for example E. coli, which can be rapidly screened by PCR for correct DNA vector constructs. Selected bacterial colonies are picked with a sterile toothpick and dabbed into the PCR master mix or sterile water. The PCR is started with an extended time at 95° C. when standard polymerase is used or with a shortened denaturation step at 100° C. and special chimeric DNA polymerase (Pavlov et al. (2006) “Thermostable DNA Polymerases for a Wide Spectrum of Applications: Comparison of a Robust Hybrid TopoTaq to other enzymes”, in Kieleczawa J: DNA Sequencing II: Optimizing Preparation and Cleanup. Jones and Bartlett, pp. 241-257).

Helicase-dependent amplification is similar to traditional PCR, but uses a constant temperature rather than cycling through denaturation and annealing/extension cycles. DNA Helicase, an enzyme that unwinds DNA, is used in place of thermal denaturation (See, Myriam et al. (2004) EMBO reports 5(8):795-800 and U.S. Pat. No. 7,282,328).

Hot-start PCR is a technique that reduces non-specific amplification during the initial set up stages of the PCR. The technique may be performed manually by heating the reaction components to the melting temperature (e.g., 95° C.) before adding the polymerase (Chou et al. (1992) Nucleic Acids Research 20:1717-1723 and U.S. Pat. Nos. 5,576,197 and 6,265,169). Specialized enzyme systems have been developed that inhibit the polymerase's activity at ambient temperature, either by the binding of an antibody (Sharkey et al. (1994) Bio/Technology 12:506-509) or by the presence of covalently bound inhibitors that only dissociate after a high-temperature activation step. Hot-start/cold-finish PCR is achieved with new hybrid polymerases that are inactive at ambient temperature and are instantly activated at elongation temperature.

Intersequence-specific (ISSR) PCR method for DNA fingerprinting that amplifies regions between some simple sequence repeats to produce a unique fingerprint of amplified fragment lengths (Zietkiewicz et al. (1994) Genomics 20(2):176-83).

Inverse PCR is a method used to allow PCR when only one internal sequence is known. This is especially useful in identifying flanking sequences to various genomic inserts. This involves a series of DNA digestions and self ligation, resulting in known sequences at either end of the unknown sequence (Ochman et al. (1988) Genetics 120:621-623 and U.S. Pat. Nos. 6,013,486; 6,106,843 or 7,132,587).

Ligation-mediated PCR uses small DNA linkers ligated to the DNA of interest and multiple primers annealing to the DNA linkers; it has been used for DNA sequencing, genome walking, and DNA footprinting (Mueller et al. (1988) Science 246:780-786).

Methylation-specific PCR (MSP) is used to detect methylation of CpG islands in genomic DNA (Herman et al. (1996) Proc Natl Acad Sci U.S.A. 93(13):9821-9826 and U.S. Pat. Nos. 6,811,982; 6,835,541 or 7,125,673). DNA is first treated with sodium bisulfite, which converts unmethylated cytosine bases to uracil, which is recognized by PCR primers as thymine. Two PCRs are then carried out on the modified DNA, using primer sets identical except at any CpG islands within the primer sequences. At these points, one primer set recognizes DNA with cytosines to amplify methylated DNA, and one set recognizes DNA with uracil or thymine to amplify unmethylated DNA. MSP using qPCR can also be performed to obtain quantitative rather than qualitative information about methylation.

Multiplex Ligation-dependent Probe Amplification (MLPA) permits multiple targets to be amplified with only a single primer pair, thus avoiding the resolution limitations of multiplex PCR (see below).

Multiplex-PCR uses of multiple, unique primer sets within a single PCR mixture to produce amplicons of varying sizes specific to different DNA sequences (See, U.S. Pat. Nos. 5,882,856; 6,531,282 or 7,118,867). By targeting multiple genes at once, additional information may be gained from a single test run that otherwise would require several times the reagents and more time to perform Annealing temperatures for each of the primer sets must be optimized to work correctly within a single reaction, and amplicon sizes, i.e., their base pair length, should be different enough to form distinct bands when visualized by gel electrophoresis.

Nested PCR increases the specificity of DNA amplification, by reducing background due to non-specific amplification of DNA. Two sets of primers are being used in two successive PCRs. In the first reaction, one pair of primers is used to generate DNA products, which besides the intended target, may still consist of non-specifically amplified DNA fragments. The product(s) are then used in a second PCR with a set of primers whose binding sites are completely or partially different from and located 3′ of each of the primers used in the first reaction (See, U.S. Pat. Nos. 5,994,006; 7,262,030 or 7,329,493). Nested PCR is often more successful in specifically amplifying long DNA fragments than conventional PCR, but it requires more detailed knowledge of the target sequences.

Overlap-extension PCR is a genetic engineering technique allowing the construction of a DNA sequence with an alteration inserted beyond the limit of the longest practical primer length.

Quantitative PCR (Q-PCR), also known as RQ-PCR, QRT-PCR and RTQ-PCR, is used to measure the quantity of a PCR product following the reaction or in real-time. See, U.S. Pat. Nos. 6,258,540; 7,101,663 or 7,188,030. Q-PCR is the method of choice to quantitatively measure starting amounts of DNA, cDNA or RNA. Q-PCR is commonly used to determine whether a DNA sequence is present in a sample and the number of its copies in the sample. The method with currently the highest level of accuracy is digital PCR as described in U.S. Pat. No. 6,440,705; U.S. Publication No. 2007/0202525; Dressman et al. (2003) Proc. Natl. Acad. Sci USA 100(15):8817-8822 and Vogelstein et al. (1999) Proc. Natl. Acad. Sci. USA. 96(16):9236-9241. More commonly, RT-PCR refers to reverse transcription PCR (see below), which is often used in conjunction with Q-PCR. QRT-PCR methods use fluorescent dyes, such as Sybr Green, or fluorophore-containing DNA probes, such as TaqMan, to measure the amount of amplified product in real time.

Reverse Transcription PCR (RT-PCR) is a method used to amplify, isolate or identify a known sequence from a cellular or tissue RNA (See, U.S. Pat. Nos. 6,759,195; 7,179,600 or 7,317,111). The PCR is preceded by a reaction using reverse transcriptase to convert RNA to cDNA. RT-PCR is widely used in expression profiling, to determine the expression of a gene or to identify the sequence of an RNA transcript, including transcription start and termination sites and, if the genomic DNA sequence of a gene is known, to map the location of exons and introns in the gene. The 5′ end of a gene (corresponding to the transcription start site) is typically identified by an RT-PCR method, named Rapid Amplification of cDNA Ends (RACE-PCR).

Thermal asymmetric interlaced PCR (TAIL-PCR) is used to isolate unknown sequence flanking a known sequence. Within the known sequence TAIL-PCR uses a nested pair of primers with differing annealing temperatures; a degenerate primer is used to amplify in the other direction from the unknown sequence (Liu et al. (1995) Genomics 25(3):674-81).

Touchdown PCR a variant of PCR that aims to reduce nonspecific background by gradually lowering the annealing temperature as PCR cycling progresses. The annealing temperature at the initial cycles is usually a few degrees (3-5° C.) above the Tₙ, of the primers used, while at the later cycles, it is a few degrees (3-5° C.) below the primer Tₘ. The higher temperatures give greater specificity for primer binding, and the lower temperatures permit more efficient amplification from the specific products formed during the initial cycles (Don et al. (1991) Nucl Acids Res 19:4008 and U.S. Pat. No. 6,232,063).

In one embodiment of the invention, probes are labeled with two fluorescent dye molecules to form so-called “molecular beacons” (Tyagi, S. and Kramer, F. R. (1996) Nat. Biotechnol. 14:303-8). Such molecular beacons signal binding to a complementary nucleic acid sequence through relief of intramolecular fluorescence quenching between dyes bound to opposing ends on an oligonucleotide probe. The use of molecular beacons for genotyping has been described (Kostrikis, L. G. (1998) Science 279:1228-9) as has the use of multiple beacons simultaneously (Marras, S. A. (1999) Genet. Anal. 14:151-6). A quenching molecule is useful with a particular fluorophore if it has sufficient spectral overlap to substantially inhibit fluorescence of the fluorophore when the two are held proximal to one another, such as in a molecular beacon, or when attached to the ends of an oligonucleotide probe from about 1 to about 25 nucleotides.

Labeled probes also can be used in conjunction with amplification of a gene or antibody of interest. (Holland et al. (1991) Proc. Natl. Acad. Sci. 88:7276-7280). U.S. Pat. No. 5,210,015 by Gelfand et al. describes fluorescence-based approaches to provide real time measurements of amplification products during PCR. Such approaches have either employed intercalating dyes (such as ethidium bromide) to indicate the amount of double-stranded DNA present, or they have employed probes containing fluorescence-quencher pairs (also referred to as the “Taq-Man” approach) where the probe is cleaved during amplification to release a fluorescent molecule whose concentration is proportional to the amount of double-stranded DNA present. During amplification, the probe is digested by the nuclease activity of a polymerase when hybridized to the target sequence to cause the fluorescent molecule to be separated from the quencher molecule, thereby causing fluorescence from the reporter molecule to appear. The Taq-Man approach uses a probe containing a reporter molecule—quencher molecule pair that specifically anneals to a region of a target polynucleotide containing the polymorphism.

Probes can be affixed to surfaces for use as “gene chips.” Such gene chips can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the invention also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley, S. O. et al. (1999) Nucleic Acids Res. 27:4830-4837.

This invention also provides for a prognostic panel of genetic markers selected from, but not limited to the genes of interest identified herein. The prognostic panel comprises probes or primers that can be used to amplify and/or for determining the molecular structure of the SP17 alone or in combination with CA125. The probes or primers can be attached or supported by a solid phase support such as, but not limited to a gene chip or microarray. The probes or primers can be detectably labeled.

In one aspect, the panel contains the herein identified probes or primers as wells as other probes or primers. In an alternative aspect, the panel includes one or more of the above noted probes or primers and others. In a further aspect, the panel consist only of the above-noted probes or primers.

Primers or probes can be affixed to surfaces for use as “gene chips” or “microarray.” Such gene chips or microarrays can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the invention also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

Various “gene chips” or “microarray” and similar technologies are know in the art. Examples of such include, but are not limited to LabCard (ACLARA Bio Sciences Inc.); GeneChip (Affymetric, Inc); LabChip (Caliper Technologies Corp); a low-density array with electrochemical sensing (Clinical Micro Sensors); LabCD System (Gamera Bioscience Corp.); Omni Grid (Gene Machines); Q Array (Genetix Ltd.); a high-throughput, automated mass spectrometry systems with liquid-phase expression technology (Gene Trace Systems, Inc.); a thermal jet spotting system (Hewlett Packard Company); Hyseq HyChip (Hyseq, Inc.); BeadArray (Illumina, Inc.); GEM (Incyte Microarray Systems); a high-throughput microarraying system that can dispense from 12 to 64 spots onto multiple glass slides (Intelligent Bio-Instruments); Molecular Biology Workstation and NanoChip (Nanogen, Inc.); a microfluidic glass chip (Orchid biosciences, Inc.); BioChip Arrayer with four PiezoTip piezoelectric drop-on-demand tips (Packard Instruments, Inc.); FlexJet (Rosetta Inpharmatic, Inc.); MALDI-TOF mass spectrometer (Sequnome); ChipMaker 2 and ChipMaker 3 (TeleChem International, Inc.); and GenoSensor (Vysis, Inc.) as identified and described in Heller (2002) Annu Rev. Biomed. Eng. 4:129-153. Examples of “Gene chips” or a “microarray” are also described in U.S. Patent Publ. Nos.: 2007/0111322, 2007/0099198, 2007/0084997, 2007/0059769 and 2007/0059765 and U.S. Pat. Nos. 7,138,506, 7,070,740, and 6,989,267.

In one aspect, “gene chips” or “microarrays” containing probes or primers for the gene of interest are provided alone or in combination with other probes and/or primers. A suitable sample is obtained from the patient extraction of genomic DNA, RNA, or any combination thereof and amplified if necessary. The DNA or RNA sample is contacted to the gene chip or microarray panel under conditions suitable for hybridization of the gene(s) of interest to the probe(s) or primer(s) contained on the gene chip or microarray. The probes or primers may be detectably labeled thereby identifying the polymorphism in the gene(s) of interest. Alternatively, a chemical or biological reaction may be used to identify the probes or primers which hybridized with the DNA or RNA of the gene(s) of interest. The genetic profile of the patient is then determined with the aid of the aforementioned apparatus and methods.

### Therapeutic Methods

This invention also provides treating a patient identified by the methods disclosed herein, as likely to have ovarian cancer, or having ovarian cancer and a poorer prognosis, such as a relative shorter overall survival time. An effective amount of a suitable therapy is administered to the patient and the prognosis of the patient can be monitored by repeating the methods as described herein. An “effective amount” is an amount sufficient to effect beneficial or desired results. An effective amount can be administered in one or more administrations, applications or dosages. Approved drugs for treatment are listed on at the web site: cancer.gov/cancertopics/druginfo/ovariancancer, last accessed on Sep. 12, 2011. Non-limiting examples of such include Adriamycin PFS; Adriamycin RDF; BEP and Carboplatin. These can be administered to subjects or individuals identified by the methods herein as suitable for the therapy, Therapeutic amounts can be empirically determined and will vary with the pathology being treated, the subject being treated and the efficacy and toxicity of the agent.

### Kits

As set forth herein, the invention provides diagnostic methods for determining the expression level of SP17 alone or in combination with CA125. In some embodiments, the methods use probes or primers comprising nucleotide sequences which are complementary to the gene of interest. Accordingly, the invention provides kits for performing these methods as well as instructions for carrying out the methods of this invention. Thus, in one aspect, this invention also provides a kit for use in identifying an ovarian cancer patient and separately, an ovarian cancer patient more likely to have tumor recurrence and/or longer or shorter overall survival, comprising, or alternatively consisting essentially of, or yet further consisting of, suitable primers or probes or a microarray for determining an expression level of SP17 alone or in combination with CA125, and instructions for use therein.

In one aspect, the components and instructions of the kit identifies a patient as more likely to experience tumor recurrence or shorter overall survival if SP17 expression level that is detected or is higher than the predetermined first value or alternatively, when CA125 and SP17 gene expression level that is present or higher than the predetermined values. In one aspect, treatment protocols and/or therapies are further provided in the kit.

In one particular aspect, the components and instructions of the kit is used to diagnose ovarian cancer in a patient, comprising or alternatively consisting essentially of, or yet further consisting of, suitable reagents to perform the methods as described herein and instructions for use. In one aspect, treatment protocols and/or therapies are further provided in the kit.

In a further aspect, the components and instructions of the kit is used to determine if the patient as less likely to experience tumor recurrence or longer overall survival time when a SP17 gene expression is absent or lower than the predetermined first value, or if CA125 level is present or higher but SP17 level is absent or lower than the predetermined values.

Briefly and for the purpose of illustration only, one of skill in the art can determine the first and second predetermined values by comparing expression values of a gene in patients with more desirable clinical parameters to those with less desirable clinical parameters. In one aspect, a predetermined value is a gene expression value that best separates patients into a group with more desirable clinical parameters and a group with less desirable clinical parameters. Such a gene expression value can be mathematically or statistically determined with methods well known in the art.

The methods are useful in the assistance of an animal, a mammal or yet further a human patient. For the purpose of illustration only, a mammal includes but is not limited to a simian, a murine, a bovine, an equine, a porcine or an ovine. The ovarian cancer can be metastatic and the cancer can be Stage I, Stage II, Stage III or Stage IV.

Suitable samples for use in the methods of the kits include, but are not limited to blood, serum, or urine or combinations thereof.

The kit can comprise at least one probe or primer which is capable of specifically hybridizing to the SP17 alone or in combination with probes and/or primer to determine the expression level of the CA125 gene and instructions for use. The kits preferably comprise at least one of the above described nucleic acids. Preferred kits for amplifying at least a portion of the gene of interest comprise two primers, at least one of which is capable of hybridizing to the allelic variant sequence. Such kits are suitable for detection of genotype by, for example, fluorescence detection, by electrochemical detection, or by other detection.

Oligonucleotides, whether used as probes or primers, contained in a kit can be detectably labeled. Labels can be detected either directly, for example for fluorescent labels, or indirectly. Indirect detection can include any detection method known to one of skill in the art, including biotin-avidin interactions, antibody binding and the like. Fluorescently labeled oligonucleotides also can contain a quenching molecule. Oligonucleotides can be bound to a surface. In one embodiment, the preferred surface is silica or glass. In another embodiment, the surface is a metal electrode.

Yet other kits of the invention comprise at least one reagent necessary to perform the assay. For example, the kit can comprise an enzyme. Alternatively the kit can comprise a buffer or any other necessary reagent.

Conditions for incubating a nucleic acid probe with a test sample depend on the format employed in the assay, the detection methods used, and the type and nature of the nucleic acid probe used in the assay. One skilled in the art will recognize that any one of the commonly available hybridization, amplification or immunological assay formats can readily be adapted to employ the nucleic acid probes for use in the present invention. Examples of such assays can be found in Chard, T. (1986) AN INTRODUCTION TO RADIOIMMUNOASSAY AND RELATED TECHNIQUES Elsevier Science Publishers, Amsterdam, The Netherlands; Bullock, G. R. et al., TECHNIQUES IN IMMUNOCYTOCHEMISTRY Academic Press, Orlando, Fla. Vol. 1 (1982), Vol. 2 (1983), Vol. 3 (1985); Tijssen, P. (1985) PRACTICE AND THEORY OF IMMUNOASSAYS: LABORATORY TECHNIQUES IN BIOCHEMISTRY AND MOLECULAR BIOLOGY, Elsevier Science Publishers, Amsterdam, The Netherlands.

The test samples used in the diagnostic kits include cells, protein or membrane extracts of cells, or biological fluids such as sputum, blood, serum, plasma, or urine. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kits can include all or some of the positive controls, negative controls, reagents, primers, sequencing markers, probes and antibodies described herein for determining the expression level of the gene of interest.

As amenable, these suggested kit components may be packaged in a manner customary for use by those of skill in the art. For example, these suggested kit components may be provided in solution or as a liquid dispersion or the like.

Methods to determine gene expression level are known in the art and briefly described herein. Non-limiting examples of these methods include a method that comprises, or alternatively consists essentially of, or yet further consists of, determining the amount of mRNA transcribed from the gene, mRNA in situ hybridization, use of gene chips or microarray, PCR, real-time PCR, or microarray. The methods are useful in the assistance of a patient such as an animal, a mammal or yet further a human patient. For the purpose of illustration only, a mammal includes but is not limited to a simian, a murine, an ovine, an equine, a canine, a bovine, a porcine or a human patient. In one aspect, the patients are stage 2 cancer patients and had not yet received any additional therapy after surgery or surgical resection. In an alternative aspect, the patients are stage 3 cancer patients and will receive or had received additional therapy after surgery or surgical resection.

Various “gene chips” or “microarray” and similar technologies are know in the art. Examples of such include, but are not limited to LabCard (ACLARA Bio Sciences Inc.); GeneChip (Affymetric, Inc); LabChip (Caliper Technologies Corp); a low-density array with electrochemical sensing (Clinical Micro Sensors); LabCD System (Gamera Bioscience Corp.); Omni Grid (Gene Machines); Q Array (Genetix Ltd.); a high-throughput, automated mass spectrometry systems with liquid-phase expression technology (Gene Trace Systems, Inc.); a thermal jet spotting system (Hewlett Packard Company); Hyseq HyChip (Hyseq, Inc.); BeadArray (Illumina, Inc.); GEM (Incyte Microarray Systems); a high-throughput microarraying system that can dispense from 12 to 64 spots onto multiple glass slides (Intelligent Bio-Instruments); Molecular Biology Workstation and NanoChip (Nanogen, Inc.); a microfluidic glass chip (Orchid biosciences, Inc.); BioChip Arrayer with four PiezoTip piezoelectric drop-on-demand tips (Packard Instruments, Inc.); FlexJet (Rosetta Inpharmatic, Inc.); MALDI-TOF mass spectrometer (Sequnome); ChipMaker 2 and ChipMaker 3 (TeleChem International, Inc.); and GenoSensor (Vysis, Inc.) as identified and described in Heller (2002) Annu Rev. Biomed. Eng. 4:129-153. Examples of “Gene chips” or a “microarray” are also described in U.S. Patent Publ. Nos.: 2007/0111322, 2007/0099198, 2007/0084997, 2007/0059769 and 2007/0059765 and U.S. Pat. Nos. 7,138,506, 7,070,740, and 6,989,267.

In one aspect, “gene chips” or “microarrays” containing probes or primers for the gene of interest are provided alone or in combination with other probes and/or primers. A suitable sample is obtained from the patient extraction of genomic DNA, RNA, or any combination thereof and amplified if necessary. The DNA or RNA sample is contacted to the gene chip or microarray panel under conditions suitable for hybridization of the gene(s) of interest to the probe(s) or primer(s) contained on the gene chip or microarray. The probes or primers may be detectably labeled thereby identifying the polymorphism in the gene(s) of interest. Alternatively, a chemical or biological reaction may be used to identify the probes or primers which hybridized with the DNA or RNA of the gene(s) of interest. The genetic profile of the patient is then determined with the aid of the aforementioned apparatus and methods.

The invention now being generally described, it will be more readily understood by reference to the following example which is included merely for purposes of illustration of certain aspects and embodiments of the present invention, and are not intended to limit the invention.

### Experimental Methods

### Human Subject Enrollment

Applicants studied a population of 323 subjects, including stage I-IV OC, benign ovarian tumors and healthy controls.

### Institutional Review Board Approval

All clinical materials were obtained with the approval from the local ethics committee (University Hospital of Groningen and Texas Tech University Health Sciences Center) and with patient consent.

### RT-PCR

Reverse-transcription PCR was performed by standard techniques as previously described^{19,20}. The primers for the SP17 transcript were 5′-GGA TCC ATG TCG ATT CCA TTC TC-3′ (SEQ ID NO: 1) and 5′-CTC GAG TCA CTT GTT TTC CTC TTT TTC-3′ (SEQ ID NO: 2).

### Immunohistochemistry

After deparaffining and re-hydration, tissue sections underwent antigen retrieval and staining as previously described^{19,21}. After counter-staining with hematoxylin (Fisher Scientific, PA, USA), pictures were taken by light microscope (Leica DMLA, IL, USA).

### ELISA for SP17

ELISA was performed by diluting 1 μL of serum in 50 μL of standard carbonate-buffered saline (coating buffer). Diluted serum was added in triplicate to a 96-well plate and incubated overnight at 4° C. Unspecific sites were saturated by incubating the plate with 1% W/V BSA in PBS for 2 hours at room temperature. BSA was removed and 50 μL mouse anti-SP17 primary antibody (developed in Applicants' laboratory and diluted 1:50 in PBS; the antibody was raised against human SP17 protein with GenBank accession No. NP 059121.1)¹³ were added, and incubated at 37° C. for 2 hours. Plates were washed thrice with PBS+0.025% Tween-20 (PBS-t) and incubated with HRP-linked anti-mouse secondary antibody (Abcam, diluted 1:4000 in PBS, 50 μL/well) for 2 hours. After 3 washing steps with PBS-t (300 μL/well), the chromogen substrate was added and allowed to incubate 5 minutes in the dark. Absorbance was then read at 405 nm. Serial dilutions of purified antigens were used to determine SP17 concentration in the serum.

### ELISA for CA125

CA125 was measured using the FDA-certified method ARCHITECT CA 125 II™ assay (Abbott Park, Ill., USA), according to the manufacturer's directions.

### Statistical Analysis

Statistical analyses were performed using GraphPad 5 statistical software (GraphPad Software, Inc., CA, USA) and setting the significance level equal to 0.05.

### Results

### Characteristics of the Study Cohorts

Applicants studied a total of 323 subjects (136 ovarian cancer patients, 45 benign ovarian tumors and 142 age-matched healthy volunteers). Subjects' distribution was as follows: 13% stage I, 6% stage II, 20% stage III, 4% stage IV, 44% healthy, 13% benign ovarian tumors. 44% of ovarian cancer were grade I/II, and 55% were grade III. Median age of ovarian cancer patients, ovarian benign tumor patients and healthy controls was 59 years. SP17 is Expressed at Different Levels in Primary Cells from Healthy Subjects, Benign Ovarian Tumors, and Ovarian Cancer Lesions.

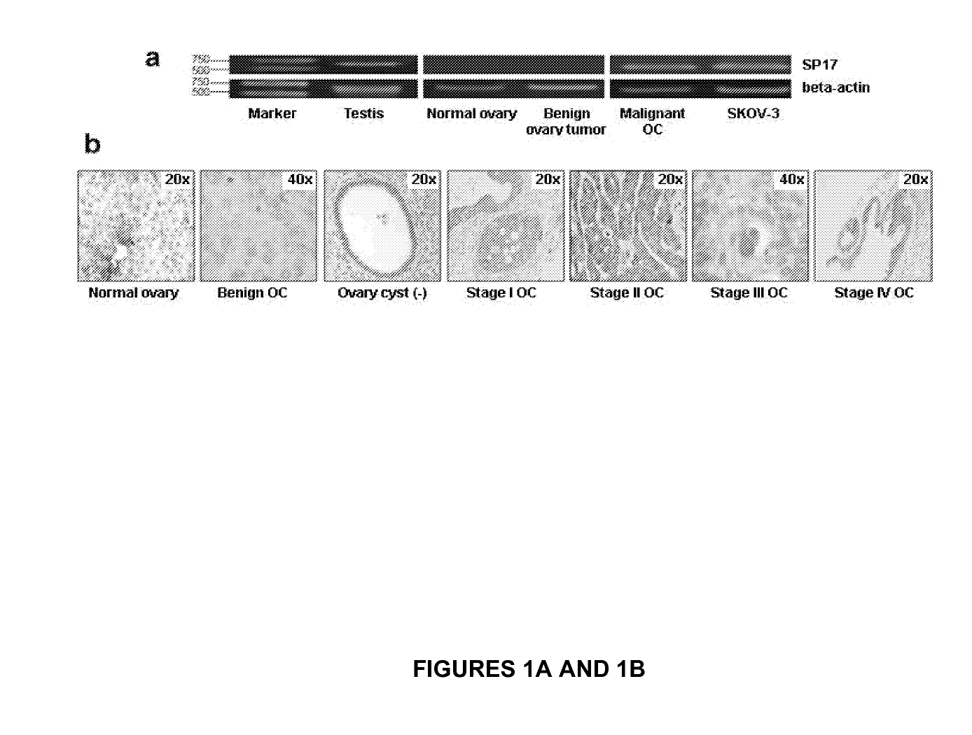

SP17 mRNA was evaluated in normal ovaries, benign ovarian tumor samples, and ovarian cancer primary cells (FIG. 1A). Positive bands were detectable in samples from ovarian cancer patients, but no transcript was present in the benign ovarian tumor samples and healthy controls. At the protein level, SP17 was detectable in all ovarian cancer samples ranging from stages I-IV, while no protein expression was shown in normal ovaries (FIG. 1B), or in any of 45 analyzed benign ovarian lesions (FIG. 1B).

Serum SP17 Concentration is Significantly Higher in Ovarian Cancer Patients Compared with Age-Matched Healthy or Benign Ovarian Tumor-Bearing Women.

Table I shows mean SP17 and CA125 serum concentration measured in healthy controls, benign ovarian tumor or ovarian cancer patients at diagnosis: mean SP17 and CA125 levels were significantly different between groups, as confirmed by one-way ANOVA. Dunn's Multiple Comparison Test revealed that SP17 and CA125 significantly lower in healthy and benign groups, as compared with ovarian cancer patients (Table I). No significant difference was detected between ovarian cancer stages, or between healthy and benign groups (p>0.05).

SP17 Serum Concentration is Associated with Overall Survival.

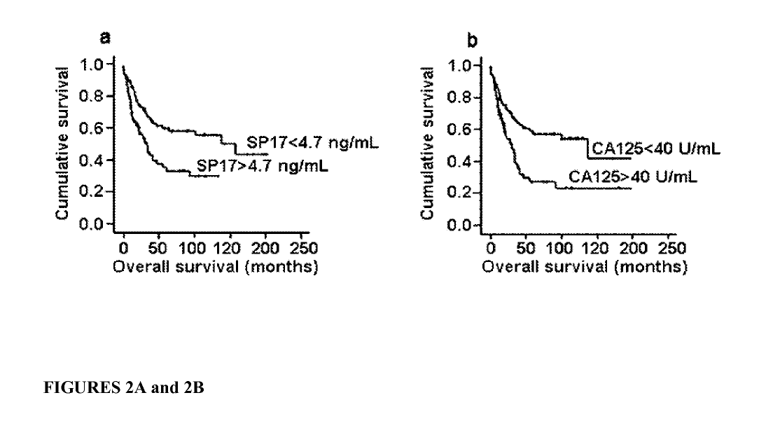

To evaluate if SP17 serum measurement could be a suitable prognostic indicator of survival time, Kaplan-Meier survival curves were built (FIGS. 2A and 2B) and the log-rank test was performed (α=0.05). Because it is known that CA125 levels are correlated with survival time, an equivalent analysis was performed with CA125 values as a positive control. Overall survival was defined as the time since primary surgery until death due to ovarian cancer or the date of last follow up. Applicants established the cut-off level of 4.7 ng/mL, as the mean SP17 serum concentration in the healthy control group plus 2 standard deviations (95^{t1} percentile). A parallel analysis was performed for CA125, yielding a cut-off value of 40 U/mL. Ovarian cancer patients were clustered in two groups, according to their SP17 values above or below 4.7 ng/mL, or CA125 values higher or lower than 40 U/mL. Both SP17 and CA125 levels were associated with overall survival time (FIGS. 2A and 2B).

SP17 Test Displays Superior Sensitivity than CA125 in Discriminating Between Early Stage Ovarian Cancer and Benign Ovarian Tumors.

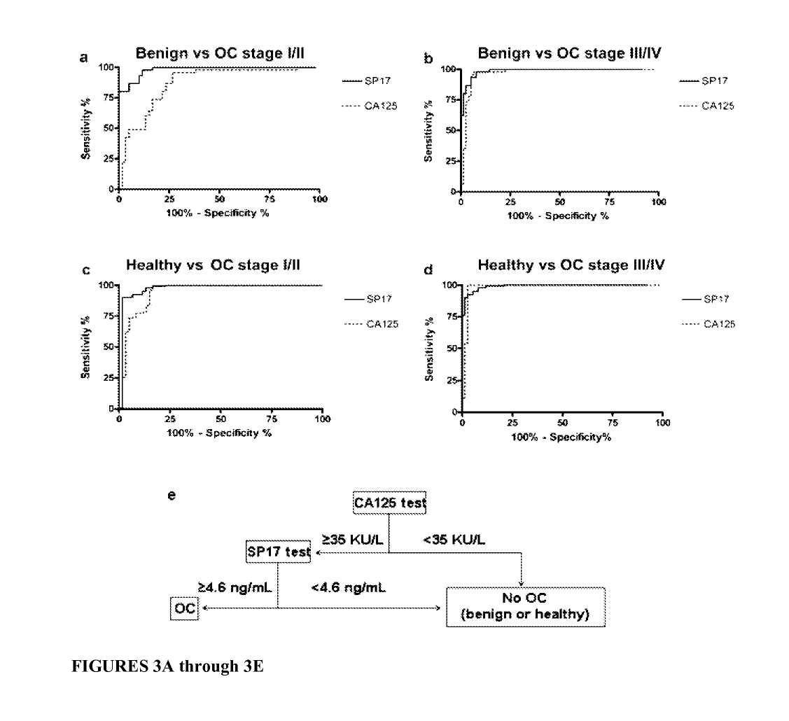

To compare SP17 and CA125 test performance in correctly diagnosing early or advanced stage ovarian cancer (stages I/II and III/IV, respectively), ROC (Receiver Operating Characteristic) curves were built (FIG. 3A to 3D) in four different settings as follows. SP17 and CA125 tests were evaluated in discriminating benign tumors from early stage or advanced stage ovarian cancer (FIGS. 3A and 3B), and healthy controls from early stage or advanced stage ovarian cancer (FIGS. 3C and 3D). When discriminating between benign tumors and stage I/II ovarian cancer, SP17 area under curve (AUC) was significantly higher than that of CA125 as compared by z-test (p<0.05). In all other conditions tested, SP17 and CA125 afforded similar AUC (p>0.05). When comparing the tests at a fixed specificity equal to 99.9%, SP17 sensitivity was 40 times higher than CA125 in discriminating benign ovarian tumors from stage VII ovarian cancer (SP17 sensitivity=88.3%, CA125 sensitivity=2.2%); no statistically significant difference in sensitivity was detected in all other tested settings.

Combined CA125 and SP17 Test Affords High Specificity and Sensitivity in Discriminating Early Stage Ovarian Cancer from Benign Ovarian Tumors or Healthy Controls.

Specificity and sensitivity of the combined CA125 and SP17 tests were calculated using different thresholds. Optimal performance of the test was obtained with 35 KU/L cut-off for CA125 and 4.6 ng/mL cut-off for SP17: FIG. 3E displays the algorithm for the combined test. The highest specificity and sensitivity of the combined test were obtained by using cut-off levels different from the ones used in the survival analysis (35 versus 40 KU/L for CA125, and 4.6 versus 4.7 ng/mL for SP17). If CA125 levels are higher than 35 KU/L, the subject is classified as potential ovarian cancer patient and undergoes SP17 evaluation. If SP17 level is higher than 4.6 ng/mL, the diagnosis is confirmed, otherwise the subject is classified as healthy or bearing a possible benign ovarian tumor. Applicants evaluated the test performance in discriminating between different groups as follows: stage VII ovarian cancer versus benign tumors, stage VII ovarian cancer versus healthy, stage VII ovarian cancer versus no malignancy (benign tumor and healthy controls). Equivalent analysis was performed for advanced stage (III/IV) ovarian cancer, and for all stages (I-IV) ovarian cancer as well. To evaluate the performance of the diagnostic test combining CA125 and SP17, specificity, sensitivity, positive predictive value (PPV) and negative predictive value (NPV) were calculated (Table II).

### DISCUSSION

This report demonstrates that the CTA SP17 is a reliable biomarker for prognostic and diagnostic purposes in ovarian cancer patients. Applicants show that it discriminates between benign ovarian tumors with good prognosis and ovarian carcinoma. Finally, Applicants provide evidence that SP17 in combination with CA125 allows for early detection of malignant lesions.

At present, ovarian cancer screening is clinically validated only when the disease prevalence is reasonably high, that is for women carrying BRCA1/2 mutations or with a family history of breast or ovarian cancer. (9) Since its discovery in 1981 as a potential biomarker to monitor the course of the disease, (22) CA125 measurement alone or together with transvaginal ultrasonography (TVU) has been widely explored as a diagnostic test for early detection of ovarian cancer, with limited success. (23-25) Increasing efforts are being made for the identification of novel tumor biomarkers to improve CA125 test specificity and sensitivity. (8) More than 30 serum biomarkers have been studied alone or in combination with CA125, (9) the most promising of which are HE4 (26), mesothelin, (27) and IL-7. (28) Serum HE4 assay, together with CA125, was reported to afford 92.9% sensitivity and 95% specificity in a cohort including healthy controls, endometriosis and ovarian cancer. (26) Based on the ovarian cancer prevalence in the cohort under evaluation, the expected PPV was 57.2%. When comparing ovarian cancer patients with healthy controls, the mesothelin test combined with CA125 had 86.5% sensitivity and 98% specificity, (27) with an expected 89.5% PPV. Applicants have previously shown that a test based on IL-7 and CA125 afforded 69% sensitivity and 100% specificity when comparing stage I-IV ovarian cancer with age-matched women carrying benign ovarian tumors, with an expected PPV of 100%. (28)

Independent analyses performed through immunohistochemistry and RT-PCR confirmed that SP17 was selectively expressed in malignant ovarian cancer. The high specificity of expression indicated that SP17 staining could be instrumental for a reliable discrimination between benign conditions and ovarian cancer in biopsy tissue samples. CTA other than SP17 have also been detected and proposed as possible prognostic markers in ovarian cancer, such as MAGE, GAGE and BAGE. (29) Recently, a number of promising non-CTA biomarkers for histological evaluation and prognosis of ovarian cancer has also been reported, such as E-Cadherin, (30), CD157 (31), steroid and xenobiotic receptor (SXR) (32), HMG-coenzyme A reductase (33), RCAS1 (34), Mammaglobin B, (35), Glypican-3 (36) and IL-7. (28) They have been shown to be associated with tumor stage or differentiation grade, but none of them afforded complete specificity. Therefore, it is likely that a panel of markers including SP17 will prove to be the best immunohistologic tool to provide the highest prognostic index in ovarian tumors. The finding that SP17 was selectively expressed only in ovarian cancer samples is consistent with previous studies, indicating that SP17 allows for tracking ovarian cancer in a murine model of the disease (14), and that it is expressed in 83% of primary ovarian tumors, but not by normal cells of the ovary. (20) Based on these results, Applicants investigated the presence of free circulating SP17 protein in the peripheral blood of ovarian cancer and benign ovarian tumor patients, or healthy controls. The data clearly indicated that serum SP17 levels were significantly higher in ovarian cancer than in healthy or benign groups. A similar result was obtained for CA125, which was used as a reference thorough the present study, since it is a validated serologic prognostic indicator. (37, 38) This is the first time that SP17 protein was analyzed in the serum of healthy individuals and cancer patients.

Survival curves show that SP17 was an independent prognostic indicator of overall survival. A critical point in the clinical decision-making process for the management of ovarian cancer is the determination of the risk of malignancy (37) basing on the risk of malignancy index (RMI) (39) or on the ACOG-SGO guidelines. (40) Because both criteria include the measurement of CA125, Applicants warrant the use of the SP17 test to improve the positive predictive value of this estimation.

Based on previous studies demonstrating that SP17 is expressed in malignant ovarian cancer cells but not in healthy ovarian tissues (12, 20), and on the prognostic validation presented here, Applicants evaluated SP17 as a novel serum biomarker for early detection of ovarian cancer by ELISA. This data show that, for the differential diagnosis of early stage ovarian cancer and benign ovarian tumors or healthy subjects, SP17 in combination with CA125 test afforded the highest PPV among the discussed serum biomarkers, with the exception of IL-7, that nevertheless yielded a lower sensitivity²⁸ compared with that of the CA125/SP17 test (69% versus 83.3%, respectively).

In conclusion, Applicants show that the measurement of SP17 serum concentration by ELISA assay affords significant discrimination between subjects with different prognoses, specifically benign ovarian tumors and ovarian carcinomas, and Applicants provide evidence that SP17 serum level is a prognostic indicator of both progression-free and overall survival. Applicants show for the first time a serum biomarker suitable for diagnosis of early stage ovarian cancer that is potentially applicable in a large-scale screening.

### TABLES

TABLE I
Measurement of SP17 and CA125 serum levels.

| | Healthy | Benign | OC | p* | p^{†} |
|---|---|---|---|---|---|
| SP17 | 3.7^{a} | 3.9 | 5.5 | P < 0.001 | P < |
| | (3.6-3.8)^{a} | (3.7-4.1) | (5.4-5.7) | | 0.001 |
| CA125 | 17.49 | 32.8 | 1,289 | P < 0.001 | P < |
| | (15.6-19.3) | (19.9-45.8) | (901.9-1,676) | | 0.001 |

Mean serum concentration (a) and 95% confidence intervals (b) of SP17 (ng/mL) and CA125 (KU/L).
p*, Dunn's test p-value for healthy versus ovarian cancer group;
p^{†}, Dunn's test p-value for benign versus OC group.

TABLE II
Performance of the combined CA125 and SP17 test. The algorithm
combining CA125 and SP17 tests results was used to calculate sensitivity
(Sens), specificity (Spec), positive predictive value (PPV) and negative
predictive value (NPV) when discriminating OC from benign
conditions or healthy conrols.

| versus | OC stage I/II | OC stage III/IV | OC all stages |
|---|---|---|---|
| Benign ovarian tumor | Sens = 83.3% | Sens = 96.0% | Sens = 90.4% |
| | Spec = 89.0% | Spec = 89.0% | Spec = 88.9% |
| | PPV = 90.9% | PPV = 92.0% | PPV = 91.6% |
| | NPV = 80.0% | NPV = 94.4% | NPV = 87.4% |
| Healthy control | Sens = 83.3% | Sens = 96.0% | Sens = 90.4% |
| | Spec = 100% | Spec = 100% | Spec = 100% |
| | PPV = 100% | PPV = 100% | PPV = 100% |
| | NPV = 93.4% | NPV = 98.4% | NPV = 96.1% |
| Benign + healthy | Sens = 83.3% | Sens = 96.0% | Sens = 88.9% |
| | Spec = 98.9% | Spec = 98.9% | Spec = 98.9% |
| | PPV = 96.1% | PPV = 96.6% | PPV = 96.4% |
| | NPV = 94.9% | NPV = 98.8% | NPV = 96.5% |

Although several embodiments of the invention are described herein in detail, it will be understood by those skilled in the art that variations may be made thereto without departing from the spirit of the invention or the scope of the appended claims.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### REFERENCES

1. Jemal A, Siegel R, Xu J, Ward E. Cancer statistics, 2010. C A Cancer J Clin 2010; 60:277-300.
2. Stany M P, Maxwell G L, Rose G S. Clinical decision making using ovarian cancer risk assessment. AJR Am J Roentgenol 2010; 194:337-42.
3. Staff A C, Bock A J, Becker C, Kempf T, Wollert K C, Davidson B. Growth differentiation factor-15 as a prognostic biomarker in ovarian cancer. Gynecol Oncol 2010; 118:237-43.
4. Berrino F, De Angelis R, Sant M, et al. Survival for eight major cancers and all cancers combined for European adults diagnosed in 1995-99: results of the EUROCARE-4 study. Lancet Oncol 2007; 8:773-83.
5. Hogg R, Friedlander M. Biology of epithelial ovarian cancer: implications for screening women at high genetic risk. J Clin Oncol 2004; 22:1315-27.
6. Hogdall E. Cancer antigen 125 and prognosis. Curr Opin Obstet Gynecol 2008; 20:4-8.
7. Nick A M, Sood A K. The ROC ‘n’ role of the multiplex assay for early detection of ovarian cancer. Nat Clin Pract Oncol 2008; 5:568-9.
8. Husseinzadeh N. Status of tumor markers in epithelial ovarian cancer has there been any progress? A review. Gynecol Oncol 2010.
9. Schorge J O, Modesitt S C, Coleman R L, et al. SGO White Paper on ovarian cancer: etiology, screening and surveillance. Gynecol Oncol 2010; 119:7-17.
10. Helzlsouer K J, Bush T L, Alberg A J, Bass K M, Zacur H, Comstock G W. Prospective study of serum CA-125 levels as markers of ovarian cancer. JAMA 1993; 269:1123-6.
11. Chiriva-Internati M, Wang Z, Salati E, Bumm K, Barlogie B, Lim S H. Sperm protein 17 (Sp17) is a suitable target for immunotherapy of multiple myeloma. Blood 2002; 100:961-5.
12. Chiriva-Internati M, Wang Z, Salati E, Timmins P, Lim S H. Tumor vaccine for ovarian carcinoma targeting sperm protein 17. Cancer 2002; 94:2447-53.
13. Chiriva-Internati M, Yu Y, Mirandola L, et al. Cancer testis antigen vaccination affords long-term protection in a murine model of ovarian cancer. PLoS One 2010; 5:e10471.
14. Chiriva-Internati M, Grizzi F, Weidanz J A, et al. A NOD/SCID tumor model for human ovarian cancer that allows tracking of tumor progression through the biomarker Sp17. J Immunol Methods 2007; 321:86-93.
15. Bumm K, Grizzi F, Franceschini B, et al. Sperm protein 17 expression defines 2 subsets of primary esthesioneuroblastoma. Hum Pathol 2005; 36:1289-93.
16. Grizzi F, Gaetani P, Franceschini B, et al. Sperm protein 17 is expressed in human nervous system tumours. BMC Cancer 2006; 6:23.
17. Gupta G, Sharma R, Chattopadhyay T K, Gupta S D, Ralhan R. Clinical significance of sperm protein 17 expression and immunogenicity in esophageal cancer. Int J Cancer 2007; 120:1739-47.
18. Li F Q, Liu Q, Han Y L, Wu B, Yin H L. Sperm protein 17 is highly expressed in endometrial and cervical cancers. BMC Cancer 2010; 10:429.
19. Chiriva-Internati M, Yu Y, Mirandola L, et al. Cancer testis antigen vaccination affords long-term protection in a murine model of ovarian cancer. PLoS One 2010; 5.
20. Straughn J M, Jr., Shaw D R, Guerrero A, et al. Expression of sperm protein 17 (Sp17) in ovarian cancer. Int J Cancer 2004; 108:805-11.
21. Li F-q, Liu Q, Han Y-l, Wu B, Yin H-l. Sperm protein 17 is highly expressed in endometrial and cervical cancers. BMC Cancer 2010; 10:429.
22. Bast R C, Jr., Klug T L, St John E, et al. A radioimmunoassay using a monoclonal antibody to monitor the course of epithelial ovarian cancer. N Engl J Med 1983; 309:883-7.
23. Stirling D, Evans D G, Pichert G, et al. Screening for familial ovarian cancer: failure of current protocols to detect ovarian cancer at an early stage according to the international Federation of gynecology and obstetrics system. J Clin Oncol 2005; 23:5588-96.
24. Hermsen B B, Olivier R I, Verheijen R H, et al. No efficacy of annual gynaecological screening in BRCA1/2 mutation carriers; an observational follow-up study. Br J Cancer 2007; 96:1335-42.
25. Kobayashi H, Yamada Y, Sado T, et al. A randomized study of screening for ovarian cancer: a multicenter study in Japan. Int J Gynecol Cancer 2008; 18:414-20.
26. Huhtinen K, Suvitie P, Hiissa J, et al. Serum H E4 concentration differentiates malignant ovarian tumours from ovarian endometriotic cysts. Br J Cancer 2009; 100:1315-9.
27. McIntosh M W, Drescher C, Karlan B, et al. Combining C A 125 and SMR serum markers for diagnosis and early detection of ovarian carcinoma. Gynecol Oncol 2004; 95:9-15.
28. Lambeck A J, Crijns A P, Leffers N, et al. Serum cytokine profiling as a diagnostic and prognostic tool in ovarian cancer: a potential role for interleukin 7. Clin Cancer Res 2007; 13:2385-91.
29. Zhang S, Zhou X, Yu H, Yu Y. Expression of tumor-specific antigen MAGE, GAGE and BAGE in ovarian cancer tissues and cell lines. BMC Cancer 2010; 10:163.
30. Dian D, Bruning A, Mylonas I. E-cadherin as a prognostic marker in human serous carcinomas of the ovary: an immunohistochemical analysis. Arch Gynecol Obstet 2010; 29:29.
31. Ortolan E, Arisio R, Morone S, et al. Functional role and prognostic significance of CD157 in ovarian carcinoma. J Natl Cancer Inst 2010; 102:1160-77.
32. Yue X, Akahira J, Utsunomiya H, et al. Steroid and Xenobiotic Receptor (SXR) as a possible prognostic marker in epithelial ovarian cancer. Pathol Int 2010; 60:400-6.
33. Brennan D J, Brandstedt J, Rexhepaj E, et al. Tumour-specific HMG-CoAR is an independent predictor of recurrence free survival in epithelial ovarian cancer. BMC Cancer 2010; 10:125.
34. Sonoda K, Miyamoto S, Kobayashi H, et al. The level of RCAS1 expression is inversely correlated with the number of vimentin-positive stromal cells in epithelial ovarian cancer. Int J Gynecol Cancer 2009; 19:838-43.
35. Tassi R A, Calza S, Ravaggi A, et al. Mammaglobin B is an independent prognostic marker in epithelial ovarian cancer and its expression is associated with reduced risk of disease recurrence. BMC Cancer 2009; 9:253.
36. Maeda D, Ota S, Takazawa Y, et al. Glypican-3 expression in clear cell adenocarcinoma of the ovary. Mod Pathol 2009; 22:824-32.
37. Stany M P, Maxwell G L, Rose G S. Clinical Decision Making Using Ovarian Cancer Risk Assessment. Am J Roentgenol 2010; 194:337-42.
38. Morris R T, Monk B J. Ovarian cancer: relevant therapy, not timing, is paramount. The Lancet 2010; 376:1120-2.
39. Geomini P, Kruitwagen R, Bremer G L, Cnossen J, Mol B W. The accuracy of risk scores in predicting ovarian malignancy: a systematic review. Obstet Gynecol 2009; 113:384-94.
40. Dearking A C, Aletti G D, McGree M E, Weaver A L, Sommerfield M K, Cliby W A. How relevant are ACOG and SGO guidelines for referral of adnexal mass? Obstet Gynecol 2007; 110:841-8.

1. A method for determining if a patient is likely to, or not likely to, experience ovarian cancer, comprising determining the expression level of the SP17 gene in a sample isolated from the patient, wherein the presence of a SP17 gene expression level or a lever higher than a predetermined first value identifies the patient as more likely to experience ovarian cancer and the absence of SP17 gene expression or SP17 gene expression lower than the predetermined first value identifies the patient as not likely to experience ovarian cancer.

2. The method of claim 1, further comprising determining the expression level of the CA125 gene is a sample isolated from the patient, wherein:
a. the presence of a SP17 level and a CA125 level or SP17 and CA125 levels higher than a predetermined first value identifies the patient as more likely to experience ovarian cancer;
b. the presence of a CA125 level or a CA125 levels higher than a predetermined first value and the absence of an SP17 level or a SP17 level lower than the predetermined first value identifies the patient as not likely to experience ovarian cancer.

3. A method for determining if an ovarian cancer patient is likely to, or not likely to, experience longer or shorter overall survival, comprising determining the expression level of the SP17 gene in a sample isolated from the patient, wherein the presence of a SP17 gene expression level or a lever higher than a predetermined first value identifies the patient as more likely to experience shorter overall survival and the absence of SP17 gene expression or SP17 gene expression lower than the predetermined first value identifies the patient as likely to experience longer overall survival.

4. The method of claim 1, further comprising determining the expression level of the CA125 gene is a sample isolated from the patient, wherein:
a. the presence of a SP17 level and a CA125 level or SP17 and CA125 levels higher than a predetermined first value identifies the patient as less likely to longer overall survival;
b. the presence of a CA125 level or a CA125 levels higher than a predetermined first value and the absence of an SP17 level or a SP17 level lower than the predetermined first value identifies the patient as more likely to longer overall survival.

5. A method for treating an ovarian cancer patient in need of treatment comprising administering to the patient an effective amount of a suitable therapy, wherein the patient is identified as in need for the treatment by a method comprising determining the expression level of the SP17 gene in a sample isolated from the patient, wherein the presence of a SP17 gene expression level or a lever higher than a predetermined first value identifies the patient as in need of the treatment.

6. The method of claim 5, further comprising determining the expression level of the CA125 gene is a sample isolated from the patient, wherein: the presence of a SP17 level and a CA125 level or SP17 and CA125 levels higher than predetermined first values identifies the patient as in need of the treatment.

7. The method of any of claim 1, 3 or 5, wherein the gene expression level is determined by a method that comprises determining the amount of mRNA transcribed from the gene and/or determining the amount of SP17 protein in the sample.

8. The method of any of claim 1, 3 or 5, wherein the gene expression level is determined by a method comprises one or more of mRNA in situ hybridization, PCR, real-time PCR, or microarray.

9. The method of any of claim 1, 3 or 5, wherein the gene expression level is determined by a method comprising detecting the polypeptide by immunohistochemistry.

10. The method of any of claim 1, 3 or 5, wherein the patient is a human patient.

11. The method of any one of claims 1, 3 or 5, wherein the sample is selected from blood, serum, or urine.

12. A kit for use in diagnosing, prognosing and/or treating an ovarian cancer patient, comprising suitable reagents for determining the expression level of the SP17 gene and optionally the expression level of CA125 gene in a patient sample, and instructions to diagnose, prognose and/or treat the patient.

13. The kit of claim 12, wherein the patient is an animal, a mammal, a bovine, an ovine, a porcine, a murine, a canine, an equine or a human patient.

14. The kit of claim 12, further comprising an amount of an effective therapy to treat the ovarian cancer patient.


SEQUENCE LISTING (2 sequences)

SEQ ID NO: 1 - DNA, 23 bp, Artificial Sequence
  Description of Artificial Sequence Synthetic primer
ggatccatgtcgattccattctc

SEQ ID NO: 2 - DNA, 27 bp, Artificial Sequence
  Description of Artificial Sequence Synthetic primer
ctcgagtcacttgttttcctctttttc